(19) United States
(12) Patent Application Publication
(10) Pub. No.: US 2016/0287209 A1
(43) Pub. Date: Oct. 6, 2016

(54) TARGET ENHANCEMENT IN IMAGING

(71) Applicant: Moshe Ein-Gal, Ramat Hasharon (IL)

(72) Inventor: Moshe Ein-Gal, Ramat Hasharon (IL)

(21) Appl. No.: 14/677,993

(22) Filed: Apr. 3, 2015

(51) Int. Cl. A61B 8/08; A61H 23/00; A61N 7/00; A61B 8/00

(52) U.S. Cl. CPC A61B 8/0833; A61B 8/54; A61H 23/008; A61N 7/00

(57) ABSTRACT

An imaging system includes a rotator operative to rotate an imaging device through at least a partial revolution about an imaging axis of the imaging device, defined as an axis along which the imaging device acquires images of an object. A processor is operative to process and average the images acquired by the imaging device.

### FIELD OF THE INVENTION

The present invention relates generally to imaging, such as ultrasonic imaging, and particularly to a method and system for target enhancement in imaging, by reducing or eliminating imaging of noise, non-target objects or artifacts.

### BACKGROUND OF THE INVENTION

As is known in the art, ultrasonic imaging uses ultrasonic energy waves that are partially reflected and partially transmitted at any interface between two media of different density. The product of material density and sonic wave velocity is known as the acoustic impedance, and the amount of reflection which occurs at the interface between two media is dependent upon the amount of change in the acoustic impedance between one medium as opposed to the other medium.

Organs, bones and other tissues in the body act as reflecting bodies within the soft tissue of the body. Likewise, any foreign inclusion acts as a reflecting body. One of the primary problems encountered in ultrasonic imaging is the difficulty to achieve reflection images of high quality resolution. The images may often be blurred or distorted to some degree, making accurate diagnosis difficult, particularly with respect to very small objects in the body. The same holds true for ultrasonic imaging in fields other than medical imaging, such as non-destructive testing of objects.

As noted before, ultrasonic images relate to spatially distributed reflections of ultrasonic energy from an object. Typical images in prior art ultrasonic imaging systems are of a planar sector symmetric about the transducer axis.

Using a spherical coordinate system (radial distance r, polar angle θ (theta) and azimuthal angle φ (phi)), an image is displaced as a sector in the (r, φ) plane for a fixed θ, wherein φ=θ=0 defines the transducer axis direction.

Since various soft tissues exhibit similar reflection coefficients, the image contrast is inherently low. A dense target like a urinary stone reflects most energy and is displayed as a brighter spot. A dark “shadow” that follows after the bright spot along the respective beam direction is due to low transmitted and reflected energy to and from the region following the dense target in the respective beam direction.

Searching for a target may be accomplished by rotating the transducer about its axis (varying the polar angle θ) until the target appears in at least one planar image. Spot brightness and associated shadow darkness may be low or be associated with low-contrast for a small target deeply located in the object, due to significant beam attenuation to/from the target, and/or due to presence of non-target objects with density similar to the target.

### SUMMARY OF THE INVENTION

The present invention seeks to provide an improved method and system for target enhancement in imaging, including target localization, by reducing or eliminating noise, non-target objects or artifacts in images, as is described more in detail hereinbelow.

An example of one of the many applications of the invention is for improved localization of urinary stones to be disintegrated by shockwaves.

There is thus provided in accordance with a non-limiting embodiment of the present invention an imaging system including a rotator operative to rotate an imaging device through at least a partial revolution about an imaging axis of the imaging device, defined as an axis along which the imaging device acquires images of an object, and a processor operative to process and average the images acquired by the imaging device.

In accordance with a non-limiting embodiment of the present invention an imaging device is rotatable by the rotator and is in communication with the processor. The at least partial revolution includes varying a polar angle theta of a rotational position of the imaging device about the imaging axis. The imaging device is operative to sequentially acquire theta-images of the object relative to different values of the polar angle theta.

In accordance with a non-limiting embodiment of the present invention the imaging device is positioned such that the imaging axis passes through a target to be imaged.

In accordance with a non-limiting embodiment of the present invention a translator is operatively linked to the imaging device to move the imaging device in translatory motion.

In accordance with a non-limiting embodiment of the present invention the translator is operative to position the imaging device such that the imaging axis passes through a target.

In accordance with a non-limiting embodiment of the present invention the imaging device includes an ultrasonic imaging device, and the imaging system further includes a flexible coupler that couples acoustic energy to and from the imaging device and the object.

In accordance with a non-limiting embodiment of the present invention a treatment device is operative to deliver treatment energy to the target.

In accordance with a non-limiting embodiment of the present invention the treatment device is operative to deliver the treatment energy with respect to an axis of symmetry collinear with the imaging axis.

In accordance with a non-limiting embodiment of the present invention a controller is in communication with the treatment device and the imaging device. The controller is operative to control a position of the imaging device in accordance with parameters of the treatment energy.

There is also provided in accordance with a non-limiting embodiment of the present invention a method of imaging including positioning the imaging device so that a target lies on the imaging axis, acquiring images of the target while rotating the imaging device through at least a partial revolution about the imaging axis by varying the polar angle theta so as to sequentially obtain theta-images of the target relative to different values of theta, and averaging the theta-images acquired by the imaging device.

### BRIEF DESCRIPTION OF THE DRAWINGS

The present invention will be understood and appreciated more fully from the following detailed description taken in conjunction with the drawings in which:

FIG. 1 is a simplified pictorial illustration of an imaging system, constructed and operative in accordance with a non-limiting embodiment of the present invention; and

FIG. 2 is a simplified flow chart of a method of imaging using the imaging system.

### DETAILED DESCRIPTION OF EMBODIMENTS

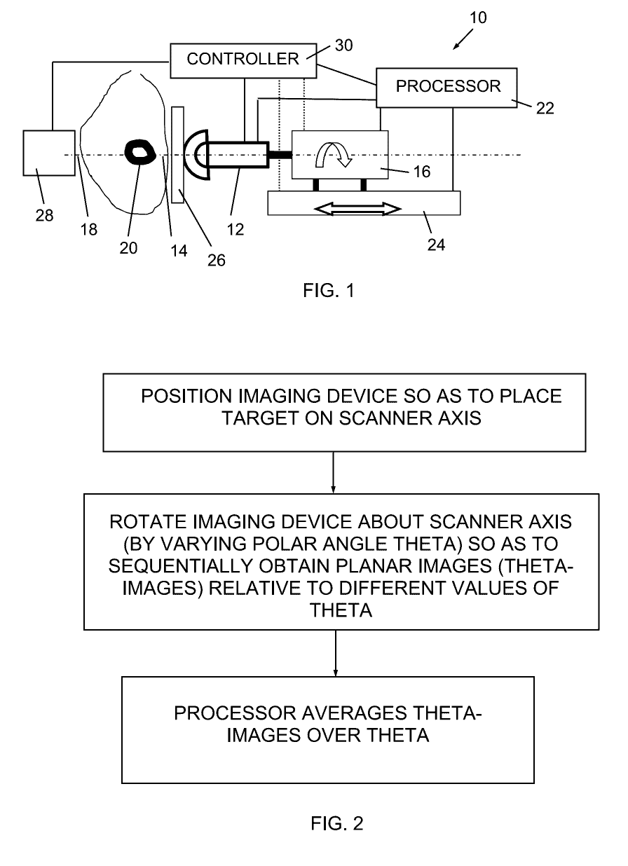

Reference is now made to FIG. 1, which illustrates imaging system 10, constructed and operative in accordance with a non-limiting embodiment of the present invention.

In one embodiment, the imaging system 10 is an accessory operatively linked to an imaging device 12, such as an ultrasonic scanner. The invention is not limited to ultrasonic imaging, and can be used for other imaging modalities, such as but not limited to, computerized tomography (CT), magnetic resonance imaging (MRI), positron emission tomography (PET) or single photon emission computed tomography (SPECT). The imaging device 12 is also referred herein as a scanner 12. Scanner 12 scans (acquires images) along a scanner axis 14 (also called imaging axis 14). The imaging axis 14 is defined as an axis along which imaging device 12 acquires images of an object.

The accessory includes a rotator 16 operative to rotate scanner 12 about scanner axis 14. In a spherical coordinate system (radial distance r, polar angle θ (theta) and azimuthal angle φ (phi)), this means rotator 16 rotates scanner 12 by varying the polar angle theta (such as a full revolution (360°), partial revolution or more than one full revolution). In this manner, scanner 12 sequentially acquires planar images (called theta-images) of an object relative to different values of theta.

Scanner 12 is positioned (as described below, or alternatively manually moved) such that the scanner axis 14 passes through a target 20 to be imaged. At this position, the scanner axis 14 is at azimuthal angle zero (phi=0).

The accessory further includes a processor 22 in communication with scanner 12. Processor 22 is capable of averaging images acquired by scanner 12. Such processors capable of processing images and averaging them are well known and require no further description to those skilled in the art.

In another embodiment, the imaging system 10 not only includes the rotator 16 and the processor 22, but also includes the imaging device 12. In all embodiments, the imaging system 10 may further include a translator 24, such as but not limited to, a linear actuator or step motor or positioning table and the like. Translator 24 is operatively linked to scanner 12 to position scanner 12 such that the scanner axis 14 passes through target 20. Alternatively or additionally, translator 24 may be operatively linked to rotator 16 which may move linearly with scanner 12. “Operatively linked” includes direct mechanical connection, such as by fasteners, indirect mechanical connection, such as by a series of gears or linking elements, and wireless communication.

As seen in FIG. 2, a method of imaging includes positioning the imaging device 12 so as to place the target 20 on the scanner axis 14 (phi=0). The imaging device 12 is rotated about scanner axis 14 (by varying polar angle theta) so as to sequentially obtain planar images (called theta-images) relative to different values of theta. Processor 22 then averages the theta-images over theta. Each of the theta-images will generally include an image of the target 20 plus an image of non-target, i.e., things around or near the target, usually of lesser importance, noise or artifacts. The average of the theta-images over theta will tend to reduce or cancel the effect of the non-target images and thus improve the target visualization. For example, the cross-correlation of objects in the theta-images generally increases with proximity of the objects to the scanner axis 14; averaging the theta-images may reduce the contrast of objects that are away from the scanner axis 14, thereby enhancing the target on the scanner axis 14.

In another embodiment, useful wherein imaging device 12 is an ultrasonic imaging device, a flexible coupler 26 is provided that couples acoustic energy to and from the scanner 12 and the object (such as target 20). Examples of flexible coupler 26 include, without limitation, a flexible membrane filled with propagating liquid, solids spacers or bellows.

In another embodiment, imaging device 12 includes a treatment device 28 operative to deliver treatment energy to target 20. Examples of treatment device 28 include, without limitation, an extracorporeal shockwave transmitter, a high-intensity ultrasonic transmitter, a radio-frequency transmitter, or a gamma irradiation system. Treatment device 28 can deliver treatment energy with respect to an axis of symmetry 18, which may be (but not necessarily) collinear with scanner axis 14. The illustrated embodiment has treatment device 28 on an opposite side of the target relative to scanner 12; however, in another embodiment treatment device 28 may be on the same side of the target as scanner 12 and treatment device 28 and scanner 12 may be incorporated in a single unit.

A controller 30 may be in communication with treatment device 28 and scanner 12. Controller 30 is operative to control the position of scanner 12 in accordance with parameters of the treatment energy, such as but not limited to, timing and intensity of pulses of the treatment energy.

What is claimed is:

1. An imaging system comprising:
a rotator operative to rotate an imaging device through at least a partial revolution about an imaging axis of the imaging device, defined as an axis along which said imaging device acquires images of an object; and
a processor operative to process and average the images acquired by the imaging device.

2. The imaging system according to claim 1, further comprising an imaging device which is rotatable by said rotator and which is in communication with said processor, and wherein said at least partial revolution comprises varying a polar angle theta of a rotational position of said imaging device about said imaging axis, and wherein said imaging device is operative to sequentially acquire theta-images of the object relative to different values of the polar angle theta.

3. The imaging system according to claim 1, wherein said imaging device is positioned such that said imaging axis passes through a target to be imaged.

4. The imaging system according to claim 1, further comprising a translator operatively linked to said imaging device to move said imaging device in translatory motion.

5. The imaging system according to claim 4, wherein said translator is operative to position said imaging device such that said imaging axis passes through a target.

6. The imaging system according to claim 1, wherein said imaging device comprises an ultrasonic imaging device, and the imaging system further comprises a flexible coupler that couples acoustic energy to and from said imaging device and the object.

7. The imaging system according to claim 3, further comprising a treatment device operative to deliver treatment energy to said target.

8. The imaging system according to claim 7, wherein said treatment device is operative to deliver the treatment energy with respect to an axis of symmetry collinear with said imaging axis.

9. The imaging system according to claim 7, further comprising a controller in communication with said treatment device and said imaging device, said controller being operative to control a position of said imaging device in accordance with parameters of the treatment energy.

10. A method of imaging comprising:
positioning the imaging device of claim 2 so that a target lies on the imaging axis;
acquiring images of the target while rotating the imaging device through at least a partial revolution about the imaging axis by varying the polar angle theta so as to sequentially obtain theta-images of the target relative to different values of theta; and
averaging the theta-images acquired by the imaging device.

11. The method according to claim 10, further comprising using a treatment device to deliver treatment energy to the target.

12. The method according to claim 11, further comprising controlling a position of said imaging device in accordance with parameters of the treatment energy.
